(19) United States
(12) Patent Application Publication
(10) Pub. No.: US 2024/0167980 A1
(43) Pub. Date: May 23, 2024

(54) APPARATUS FOR MEASURING CAPSAICIN IN CHILI PEPPERS

(71) Applicant: DAEPUNGNYUN CO., LTD., Jeollabuk-do (KR)

(72) Inventors: Hyeong Won SEO, Jeongeup-si, Jeollabuk-do (KR); Chang Woo KIM, Jeonju-si, Jeollabuk-do (KR); Myoung Sun SHIN, Jeonju-si, Jeollabuk-do (KR); Gyeong Sook BANG, Jeonju-si Jeollabuk-do (KR); Ki Wan KIM, Jeonju-si, Jeollabuk-do (KR); Pan Su JANG, Daejeon (KR); Woo Hyeung CHO, Daejeon (KR)

(21) Appl. No.: 17/772,655

(22) PCT Filed: Mar. 4, 2022
(86) PCT No.: PCT/KR2022/003126

§ 371 Date: Apr. 28, 2022

(51) Int. Cl. G01N 27/48; G01N 27/416; G01N 33/00; G01N 33/02

(52) U.S. Cl. CPC G01N 27/48; G01N 27/4163; G01N 33/025; G01N 2033/0091

(57) ABSTRACT

Proposed is an apparatus for measuring capsaicin in chili peppers. More particularly, proposed are an apparatus and a method for measuring capsaicin in chili peppers, the apparatus and the method being capable of measuring the capsaicin content of chili peppers simply and precisely. The apparatus includes: a first electrochemical measuring cell and a second electrochemical measuring cell into each of which a capsaicin extract is injected; a multiplexer electrically connected to a selected one of the first and second electrochemical measuring cells; a potentiostat applying voltage to the first or second electrochemical measuring cell and measuring an oxidation current value; and a controller controlling the potentiostat and the multiplexer to sequentially electrically connect the potentiostat to each of the first and second electrochemical measuring cells and measuring a first capsaicin content and a second capsaicin content on the basis of the oxidation current value as a function of applied voltage.

### TECHNICAL FIELD

The present disclosure relates generally to an apparatus for measuring capsaicin in chili peppers. More particularly, the present disclosure relates to an apparatus and a method for measuring capsaicin in chili peppers, the apparatus and the method being capable of measuring the capsaicin content of chili peppers simply and precisely.

### BACKGROUND ART

Capsaicin and dihydrocapsaicin, which are responsible for approximately 90% of the total pungency of chili peppers, are derivatives of capsaicinoids and are known as endogenous fluorescence materials.

The industry related to chili peppers relies on subjective sensory evaluation to measure the capsaicin content of chili peppers and chili pepper powder. Thus, there is a problem of causing unnecessary logistics costs associated with product returns caused by subjective sensory differences in capsaicin content in transactions of chili peppers and chili pepper powder.

Meanwhile, as a method for measuring the content of capsaicin, a pungent component of chili pepper, Patent Document 0001 discloses a method involving extracting capsaicin from chili pepper using an extraction solvent such as acetone and then quantifying capsaicin using high-performance liquid chromatography (HPLC).

However, such HPLC method is costly and requires specialized knowledge to perform, so there is a limit to self-measurement by companies themselves.

### DISCLOSURE

### Technical Problem

Accordingly, the present disclosure has been made keeping in mind the above problems occurring in the related art, and an objective of the present disclosure is to provide an apparatus for measuring capsaicin in chili peppers, the apparatus being capable of simply and precisely measuring the capsaicin content of chili peppers at a low cost.

### Technical Solution

In order to accomplish the above objective, the present disclosure provides an apparatus for measuring capsaicin in chili pepper powder, the apparatus including:

a first electrochemical measuring cell and a second electrochemical measuring cell into each of which a capsaicin extract extracted from chili pepper powder is injected;

a multiplexer electrically connected to a selected one of the first electrochemical measuring cell and the second electrochemical measuring cell;

a potentiostat configured to apply a voltage to the first electrochemical measuring cell or the second electrochemical measuring cell using cyclic voltammetry through the multiplexer and measure an oxidation current value; and

a controller configured to control the potentiostat and the multiplexer to sequentially electrically connect the potentiostat to each of the first electrochemical measuring cell and the second electrochemical measuring cell, and configured to measure a first capsaicin content and a second capsaicin content on the basis of the oxidation current value as a function of applied voltage of each of the first electrochemical measuring cell and the second electrochemical measuring cell, the value being input by the potentiostat.

A first input port and a second input port electrically connected to the multiplexer may be provided, and each of the first electrochemical measuring cell and the second electrochemical measuring cell may be provided with a connector, the respective connectors being connected to the first input port and the second input port.

Each of the first electrochemical measuring cell and the second electrochemical measuring cell may include: a measuring cell body at which the connector is installed; and an electrochemical sensor disposed in the measuring cell body, and having a working electrode, a reference electrode, and a counter electrode formed at a second side thereof and a connection terminal formed at a first side thereof and connected to the connector.

The measuring cell body may include: a lower body having an insertion groove that is formed at a second side of an upper surface thereof and allows the electrochemical sensor to be inserted thereinto, and the connector that is installed at a first side thereof; an upper body detachably coupled to the lower body, and having a central cell portion vertically passing therethrough and an annular groove formed in a bottom surface thereof around the cell portion; and a ring-shaped sealing member inserted into the annular groove.

The controller may include: a computing unit configured to measure the first capsaicin content and the second capsaicin content on the basis of the oxidation current value as a function of voltage sequentially measured by each of the first electrochemical measuring cell and the second electrochemical measuring cell and input by the potentiostat; a reliability determining unit configured to determine measurement reliability by calculating an average value, standard deviation, and coefficient of variation (%) of the first capsaicin content and the second capsaicin content and then comparing the calculated coefficient of variation with a reference coefficient of variation; and an information outputting unit configured to output capsaicin content information to a display when reliability of measurement results is approved by the reliability determining unit.

The computing unit may measure the first capsaicin content and the second capsaicin content by comparing, with a calibration curve, a peak area calculated from a voltammogram of the digitized oxidation current value as a function of voltage obtained from the first electrochemical measuring cell and a peak area calculated from a voltammogram of the digitized oxidation current value as a function of voltage obtained from the second electrochemical measuring cell.

### Advantageous Effects

An apparatus for measuring capsaicin in chili pepper powder according to the present disclosure can measure each oxidation current value as a function of applied voltage sequentially for capsaicin extracts injected into a first electrochemical measuring cell and a second electrochemical measuring cell, thereby enabling accurate measurement of the capsaicin content of chili pepper powder within a short period of time.

In particular, a reliability determining unit can determine measurement reliability using the coefficient of variation of a first capsaicin content of the first electrochemical measuring cell and a second capsaicin content of the second electrochemical measuring cell. Thus, it is possible to enable reliable measurement of the capsaicin content.

### DESCRIPTION OF DRAWINGS

FIG. 1 is a view schematically illustrating an apparatus for measuring capsaicin in chili pepper powder according to the present disclosure.

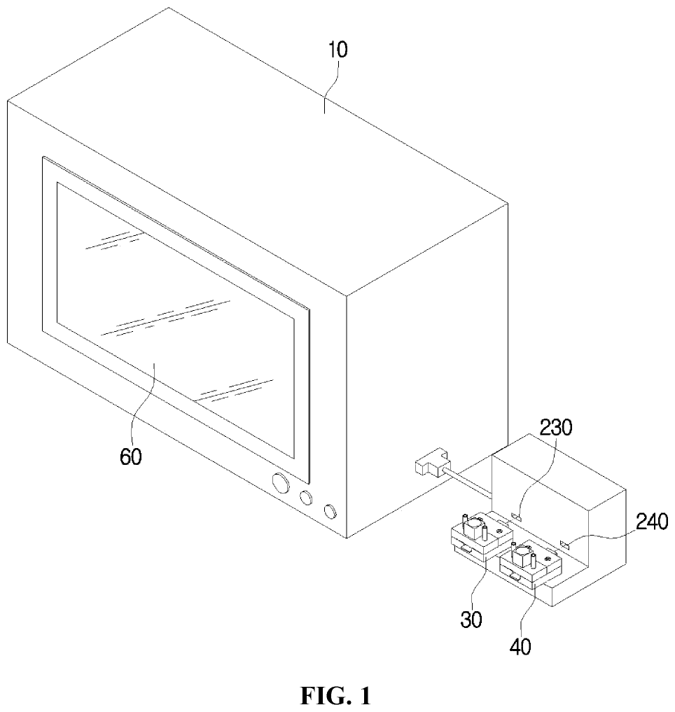

FIG. 2 is a block diagram schematically illustrating the configuration of the apparatus for measuring capsaicin in chili pepper powder according to the present disclosure.

FIG. 3 is a view schematically illustrating a first electrochemical measuring cell or a second electrochemical measuring cell.

FIG. 4 is a view schematically illustrating an electrochemical measuring cell from which an upper body is removed.

FIG. 5 is a longitudinal sectional view schematically illustrating a cross-section taken along line A-A of FIG. 3.

### BEST MODE

Hereinafter, an apparatus and a method for measuring capsaicin in chili peppers according to the present disclosure will be described in detail with reference to the drawings.

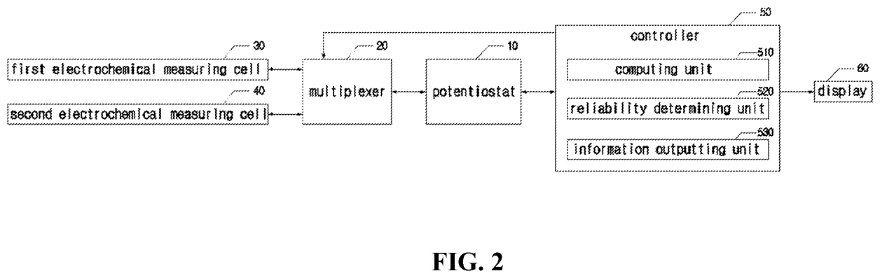

FIG. 2 is a block diagram schematically illustrating the configuration of an apparatus for measuring capsaicin in chili pepper powder according to the present disclosure. FIG. 2 is a block diagram schematically illustrating the configuration of the apparatus for measuring capsaicin in chili pepper powder according to the present disclosure.

The apparatus for measuring capsaicin in chili peppers according to the present disclosure may largely include a potentiostat 10, a multiplexer 20, a first electrochemical measuring cell 30, a second electrochemical measuring cell 40, a controller 50, and a display 60.

First, the potentiostat 10 measures an oxidation current value of an capsaicin extract by applying a constant potential difference between a working electrode of each of the first electrochemical measuring cell 30 and the second electrochemical measuring cell 40, where electrochemical reactions take place, and a reference electrode thereof.

The potentiostat 10 generates a constant potential difference between the working electrode and the reference electrode of each of the first electrochemical measuring cell 30 and the second electrochemical measuring cell 40 through a DAC. An oxidation current is induced from the working electrode of each of the first electrochemical measuring cell 30 and the second electrochemical measuring cell 40 to a counter electrode thereof. The thus induced oxidation current is digitized by an ADC, and the thus digitized oxidation current value is input to the controller 50.

The multiplexer 20 electrically connects a selected one of the first electrochemical measuring cell 30 and the second electrochemical measuring cell 40 to the potentiostat 10.

In the present disclosure, in order to simply and precisely measure the capsaicin content of chili peppers, the capsaicin content is measured by the single potentiostat 10 sequentially for each of the first electrochemical measuring cell 30 and the second electrochemical measuring cell 40 into each of which a capsaicin extract extracted from chili peppers and chili pepper powder is injected. To this end, each of the first electrochemical measuring cell 30 and the second electrochemical measuring cell 40 is sequentially electrically connected to the potentiostat 10 using the multiplexer 20.

The multiplexer 20 electrically connects the first electrochemical measuring cell 30 or the second electrochemical measuring cell 40 to the potentiostat 10 in response to a channel control signal from the controller 50.

In order to conveniently connect the multiplexer 20 to the first electrochemical measuring cell 30 and the second electrochemical measuring cell 40, it is preferable that a first input port 230 and a second input port 240 electrically connected to the multiplexer 20 are provided. Specifications of the first input port 230 and the second input port 240 are not particularly limited, and USB type ports may be used.

In addition, each of the first electrochemical measuring cell 30 and the second electrochemical measuring cell 40 may be provided at an end portion thereof with a connector 314, the respective connectors being connected to the first input port 230 and the second input port 240.

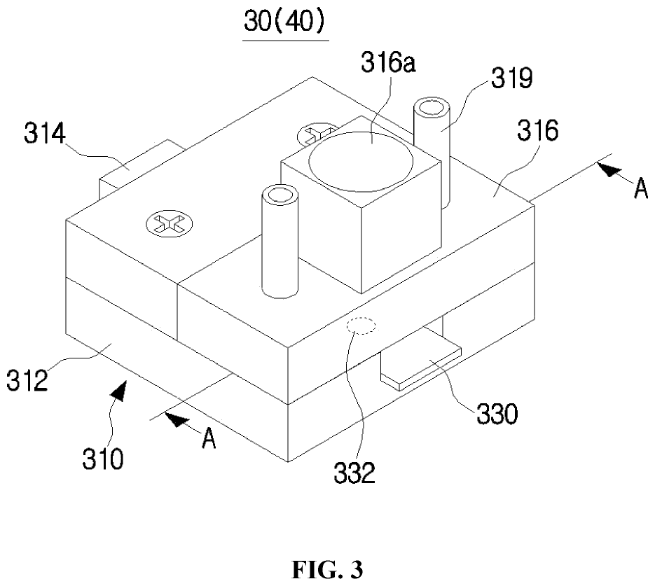

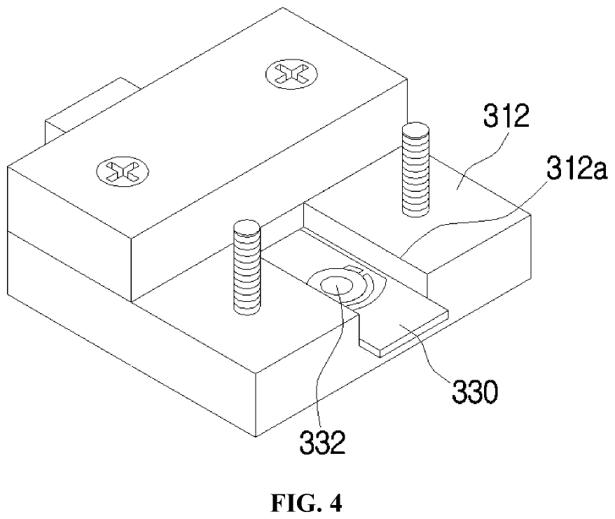

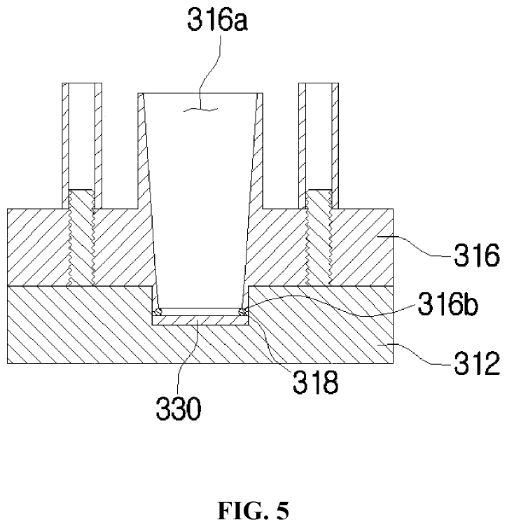

FIG. 3 is a view schematically illustrating the first electrochemical measuring cell 30 or the second electrochemical measuring cell 40. FIG. 4 is a view schematically illustrating an electrochemical measuring cell from which an upper body 316 is removed. FIG. 5 is a longitudinal sectional view schematically illustrating a cross-section taken along line A-A of FIG. 3.

Each of the first electrochemical measuring cell 30 and the second electrochemical measuring cell 40 allows the capsaicin extract extracted from chili pepper powder to be injected thereinto and measures an oxidation current induced to the counter electrode according to a voltage applied to the working electrode.

Each of the first electrochemical measuring cell 30 and the second electrochemical measuring cell 40 includes a measuring cell body 310 and an electrochemical sensor 330 disposed in the measuring cell body 310.

The measuring cell body 310 is provided at an end portion thereof with the connector 314 electrically connected to electrodes of the electrochemical sensor 330 and inserted into and connected to the first input port 230 or the second input port 240 in order to improve the convenience of use of the first electrochemical measuring cell 30 and the second electrochemical measuring cell 40.

The electrochemical sensor 330 has a working electrode, a reference electrode, and a counter electrode formed at a second side thereof, and a connection terminal formed at a first side thereof and connected to the working electrode, the reference electrode, and the counter electrode.

The measuring cell body 310 includes: a lower body 312 having an insertion groove 312a that is formed at a second side of an upper surface thereof and allows the electrochemical sensor 330 to be inserted thereinto; the connector 314 installed at a first side of the lower body 312; the upper body 316 detachably coupled to the lower body 312, and having a central cell portion 316a vertically passing therethrough and an annular groove 316b formed in a bottom surface thereof around the cell portion 316a; and a ring-shaped sealing member 318 inserted into the annular groove 316b.

The connector 314 is provided at a side thereof with a connection portion (not illustrated) to which the connection terminal of the electrochemical sensor 330 inserted into the insertion groove 312a of the lower body 312 is connected.

The upper body 316 is coupled to the lower body 312 by a fastening member 319 in a state in which the electrochemical sensor 330 is inserted into the insertion groove 312a of the lower body 312. The cell portion 316a of the upper body 316 is formed at a position corresponding to the position of the electrodes of the electrochemical sensor 330 inserted into the insertion groove 312a of the lower body 312.

In this case, as the upper body 316 is coupled to the lower body 312 by the fastening member in a state in which the sealing member 318 is inserted into the bottom surface of the upper body 316 around the cell portion 316a, the sealing member 318 surrounds and is in close contact with the electrodes of the electrochemical sensor 330 to maintain watertightness. Thus, when injected through the cell portion 316a of the upper body 316, the capsaicin extract is accommodated on the electrodes 332 of the electrochemical sensor 330 without the possibility of flowing into the lower body 312, the connector 314, etc., thereby making it possible to measure the capsaicin content more precisely.

The controller 50 controls the potentiostat 10 and the multiplexer 20, calculates a peak area on the basis of the digitized oxidation current value as function of applied voltage input by the ADC of the potentiostat 10, and calculates the capsaicin content by comparing the calculated value with a calibration curve.

The controller 50 controls the potentiostat 10 to apply a voltage to the first electrochemical measuring cell 30 and the second electrochemical measuring cell 40 using cyclic voltammetry and controls the multiplexer 20 to sequentially electrically connect the potentiostat 10 to each of the first electrochemical measuring cell 30 and the second electrochemical measuring cell 40.

In addition, the controller 50 includes: a computing unit 510 for measuring a first capsaicin content and a second capsaicin content on the basis of the digitized oxidation current value as function of voltage measured sequentially by each of the first electrochemical measuring cell 30 and the second electrochemical measuring cell 40 and input by the ADC of the potentiostat 10; a reliability determining unit 520 for determining measurement reliability by calculating an average value, standard deviation, and coefficient of variation (%) of the first capsaicin content and the second capsaicin content and comparing the calculated coefficient of variation with a reference coefficient of variation; and an information outputting unit 530 for outputting capsaicin content information to the display 60 when the reliability of measurement results is approved by the reliability determining unit 520.

In particular, the computing unit 510 measures the first capsaicin content and the second capsaicin content by comparing, with a calibration curve, a peak area calculated from a voltammogram of the digitized oxidation current value as a function of voltage obtained from the first electrochemical measuring cell 30 and a peak area calculated from a voltammogram of the digitized oxidation current value as a function of voltage obtained from the second electrochemical measuring cell 40.

The calibration curve may be obtained by calculating a peak area from a voltammogram of an oxidation current value as a function of voltage measured for a plurality of capsaicin standard solutions with different concentrations using cyclic voltammetry, and creating the calibration curve using a linear regression equation on the basis of the peak area of the plurality of capsaicin standard solutions with different concentrations.

For example, in a case where the reference coefficient of variation is set to “less than 10%”, when the coefficient of variation for the first capsaicin content and the second capsaicin content is calculated as 5.2%, the reliability of measurement results is approved and the information outputting unit 530 outputs the capsaicin content information to the display 60. In addition, when the coefficient of variation is calculated as 10.8%, the reliability of measurement results is not approved and the information outputting unit 530 outputs a measurement error to the display 60. As such, by determining the measurement reliability by the reliability determining unit 520, it is possible to measure the capsaicin content more precisely.

Next, a method of measuring capsaicin in chili peppers using the apparatus for measuring capsaicin in chili peppers will be described as follows.

The method of measuring capsaicin in chili peppers largely includes a capsaicin extraction step, a capsaicin extract injection step, a first oxidation current value measurement step, a second oxidation current value measurement step, a first and second capsaicin content measurement step, a reliability determination step, and a capsaicin content output step.

First, the capsaicin extraction step is a step of extracting chili pepper powder with an organic solvent and then removing impurities to obtain a capsaicin extract. The method of obtaining the capsaicin extract is not particularly limited, and a method of obtaining a capsaicin extract by extracting capsaicin by ultrasonic extraction with ethanol and then removing impurities may be used.

Then, the capsaicin extract injection step is a step of injecting the capsaicin extract into each of the first electrochemical measuring cell 30 and the second electrochemical measuring cell 40 electrically connected to the potentiostat 10 of the apparatus.

The electrochemical sensor 330 having the working electrode, the reference electrode, and the counter electrode is provided in each of the first electrochemical measuring cell 30 and the second electrochemical measuring cell 40.

The first oxidation current value measurement step is a step of measuring, by the potentiostat 10, a first oxidation current value as a function of applied voltage for the capsaicin extract injected into the first electrochemical measuring cell 30 using cyclic voltammetry.

At this time, in a state in which the controller 50 controls the potentiostat 10 and the multiplexer 20 to electrically connect the potentiostat 10 and the first electrochemical measuring cell 30, the potentiostat 10 measures the first oxidation current value as a function of applied voltage for the capsaicin extract injected into the first electrochemical measuring cell 30 using cyclic voltammetry.

Then, the second oxidation current value measurement step is a step of measuring, by the potentiostat 10, a second oxidation current value as a function of applied voltage for the capsaicin extract injected into the second electrochemical measuring cell 40 using cyclic voltammetry.

At this time, in a state in which the controller 50 controls the potentiostat 10 and the multiplexer 20 to electrically connect the potentiostat 10 and the second electrochemical measuring cell 40, the potentiostat 10 measures the second oxidation current value as a function of applied voltage for the capsaicin extract injected into the second electrochemical measuring cell 40 using cyclic voltammetry.

Then, the first and second capsaicin content measurement step is a step of measuring a first capsaicin content and a second capsaicin content by calculating respective peak areas from a voltammogram of the first oxidation current value as a function of applied voltage and a voltammogram of the second oxidation current value as a function of applied voltage, and then comparing the calculated peak area values with a calibration curve.

Then, the reliability determination step is a step of determining measurement reliability by calculating an average value, standard deviation, and coefficient of variation of the first capsaicin content and the second capsaicin content, and then comparing the calculated coefficient of variation with a reference coefficient of variation.

In other words, when the calculated coefficient of variation is less than the reference coefficient of variation, the measurement reliability of the measurement result is approved, and on the other hand, when the coefficient of variation is equal to or larger than the reference coefficient of variation, the reliability of measurement results is not approved.

Then, the capsaicin content output step is a step of outputting information on the first capsaicin content and the second capsaicin content to the display 60 when the coefficient of variation is determined to fall within the reference coefficient of variation range in the reliability determination step.

Here, the information on the first capsaicin content and the second capsaicin content includes the first capsaicin content, the second capsaicin content, the average value, the standard deviation, and the coefficient of variation (%), and the like.

### INDUSTRIAL APPLICABILITY

An apparatus for measuring capsaicin in chili pepper powder according to the present disclosure can measure each oxidation current value as a function of applied voltage sequentially for capsaicin extracts injected into a first electrochemical measuring cell and a second electrochemical measuring cell, thereby enabling accurate measurement of the capsaicin content of chili pepper powder within a short period of time.

In particular, a reliability determining unit can determine measurement reliability using the coefficient of variation of a first capsaicin content of the first electrochemical measuring cell and a second capsaicin content of the second electrochemical measuring cell. Thus, it is possible to enable reliable measurement of the capsaicin content.

1. An apparatus for measuring capsaicin in chili pepper powder, the apparatus comprising:
a first electrochemical measuring cell and a second electrochemical measuring cell into each of which a capsaicin extract extracted from chili pepper powder is injected;
a multiplexer electrically connected to a selected one of the first electrochemical measuring cell and the second electrochemical measuring cell;
a potentiostat configured to apply a voltage to the first electrochemical measuring cell or the second electrochemical measuring cell using cyclic voltammetry through the multiplexer and measure an oxidation current value; and
a controller configured to control the potentiostat and the multiplexer to sequentially electrically connect the potentiostat to each of the first electrochemical measuring cell and the second electrochemical measuring cell, and configured to measure a first capsaicin content and a second capsaicin content on the basis of the oxidation current value as a function of applied voltage of each of the first electrochemical measuring cell and the second electrochemical measuring cell, the value being input by the potentiostat.

2. The apparatus of claim 1, wherein a first input port and a second input port electrically connected to the multiplexer are provided, and each of the first electrochemical measuring cell and the second electrochemical measuring cell is provided with a connector, the respective connectors being connected to the first input port and the second input port.

3. The apparatus of claim 2, wherein each of the first electrochemical measuring cell and the second electrochemical measuring cell comprises:
a measuring cell body at which the connector is installed; and
an electrochemical sensor disposed in the measuring cell body, and having a working electrode, a reference electrode, and a counter electrode formed at a second side thereof and a connection terminal formed at a first side thereof and connected to the connector.

4. The apparatus of claim 3, wherein the measuring cell body comprises:
a lower body having an insertion groove that is formed at a second side of an upper surface thereof and allows the electrochemical sensor to be inserted thereinto, and the connector that is installed at a first side thereof;
an upper body detachably coupled to the lower body, and having a central cell portion vertically passing therethrough and an annular groove formed in a bottom surface thereof around the cell portion; and
a ring-shaped sealing member inserted into the annular groove.

5. The apparatus of claim 1, wherein the controller comprises:
a computing unit configured to measure the first capsaicin content and the second capsaicin content on the basis of the oxidation current value as a function of voltage sequentially measured by each of the first electrochemical measuring cell and the second electrochemical measuring cell and input by the potentiostat;
a reliability determining unit configured to determine measurement reliability by calculating an average value, standard deviation, and coefficient of variation (%) of the first capsaicin content and the second capsaicin content and then comparing the calculated coefficient of variation with a reference coefficient of variation; and
an information outputting unit configured to output capsaicin content information to a display when reliability of measurement results is approved by the reliability determining unit.

6. The apparatus of claim 5, wherein the computing unit measures the first capsaicin content and the second capsaicin content by comparing, with a calibration curve, a peak area calculated from a voltammogram of the digitized oxidation current value as a function of voltage obtained from the first electrochemical measuring cell and a peak area calculated from a voltammogram of the digitized oxidation current value as a function of voltage obtained from the second electrochemical measuring cell.
